(19) United States
(12) Patent Application Publication
(10) Pub. No.: US 2006/0281901 A1
(43) Pub. Date: Dec. 14, 2006

(54) Method for the production of amyloid-beta peptide using ubiquitin

(76) Inventors: Yung Joon Yoo, Gwangju (KR); Eun Kyung Lee, Gwangju (KR)

Correspondence Address: David A. Einhom, Esq.;Anderson Kill & Olick, P.C., 1251 Avenue of the Americas, New York, NY, 10020, US

(21) Appl. No.: 11/325,390

(22) Filed: Jan. 3, 2006

(30) Foreign Application Priority Data
Jun. 14, 2005 (KR) 10-2005-0050824

(51) Int. Cl. C07K 14/47; C07H 21/04; C12P 21/06; C12N 15/74; C12N 1/21

(52) U.S. Cl. 530/350; 435/69.1; 435/471; 435/252.3; 536/23.5

(57) ABSTRACT

The method for the production of amyloid-β peptide using ubiquitin as a fusion partner according to the present invention overcomes such problems of chemical methods for synthesizing amyloid-β peptide as low yield and high production cost, and can be effectively used for producing amyloid-β peptide in a high yield.

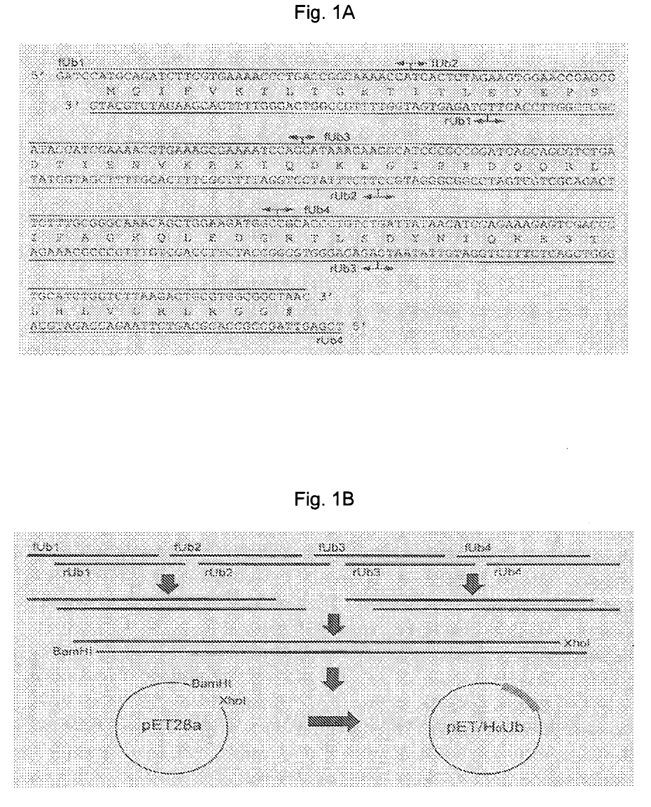

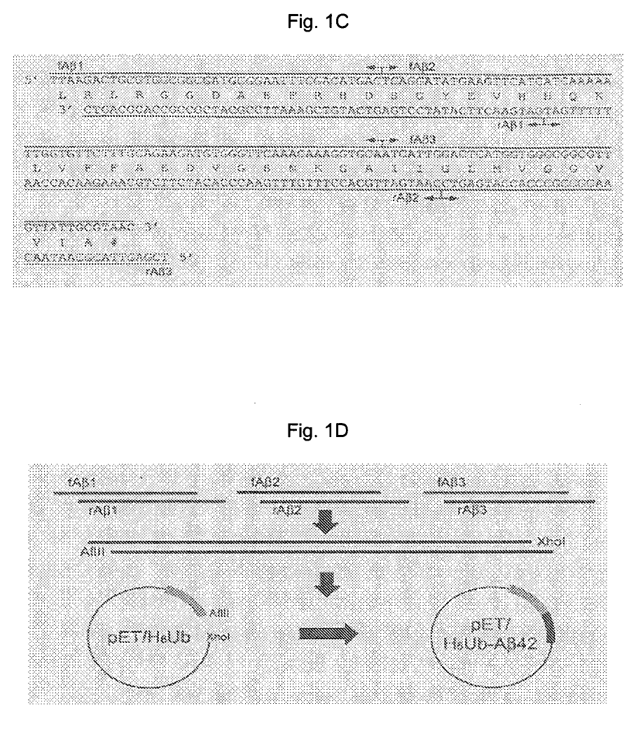

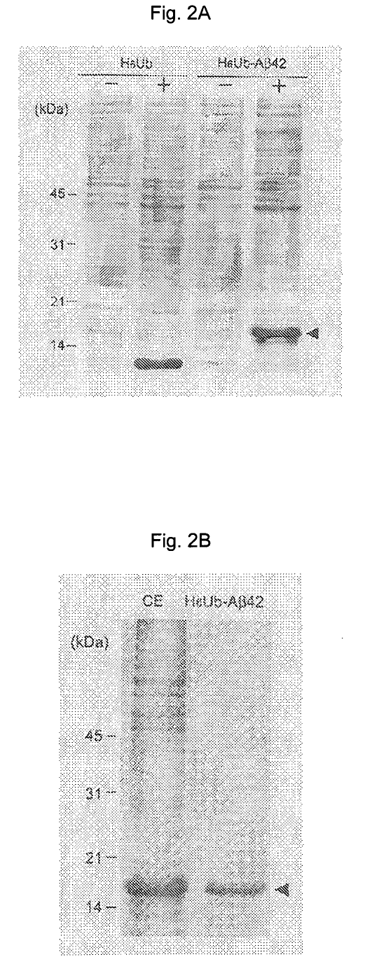

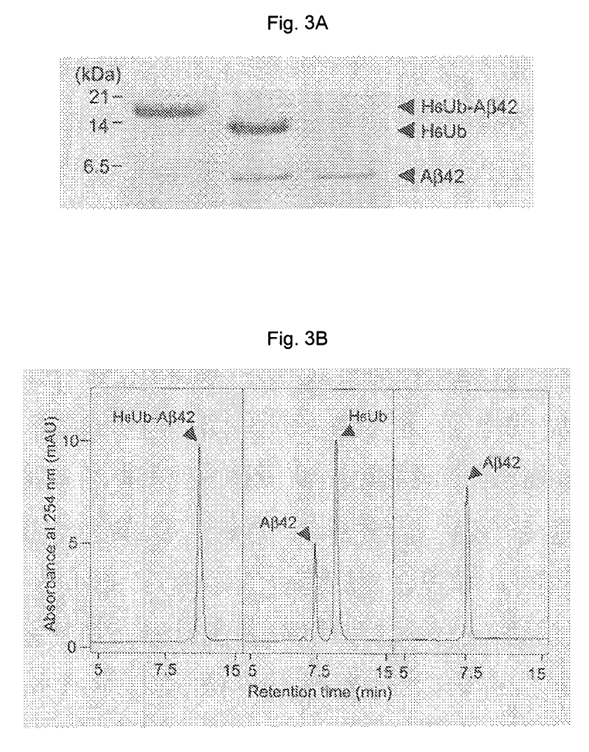

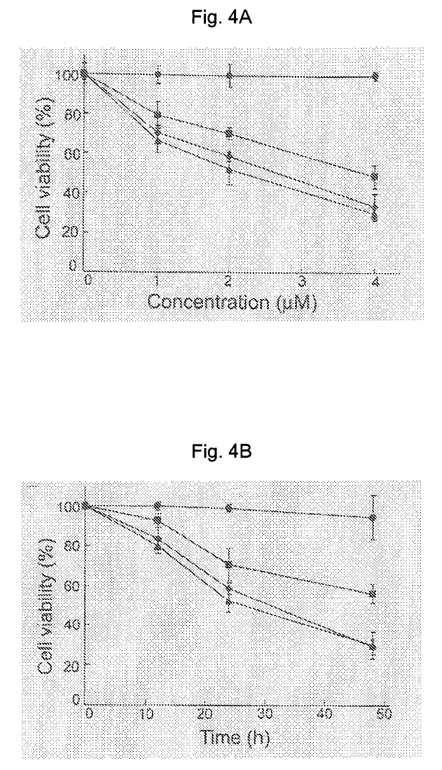

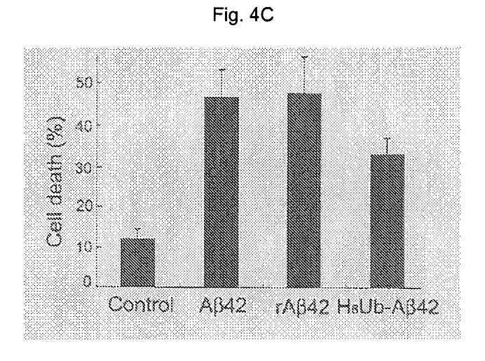

### FIELD OF THE INVENTION

The present invention relates to a method for the production of amyloid-β peptide using ubiquitin as a fusion partner.

### BACKGROUND OF THE INVENTION

Amyloid-β peptide (Aβ) is the principal component of senile plaques commonly found in the brain of Alzheimer's disease patients and it consists of 40, 42 or 43 amino acids derived by proteolytic cleavage of amyloid precursor protein (APP). When abnormally accumulated in the brain, the amyloid-β peptide shows neurotoxicity such as neuronal degeneration, nerve cell death and synapse loss at the end of dead nerve cells, which leads to a degenerative disorder clinically characterized by progressive loss of memory, temporal and local orientation cognition, reasoning, judgment and emotional stability. However, the precise mechanism of the abnormal accumulation of amyloid-β peptide in the brain or the relationship between the formation of pathological amyloid-β peptide and the pathogenesis of Alzheimer's disease has not been distinctively established.

Accordingly, there is a need to study the cytotoxic mechanism of amyloid-β peptide and its role in the pathogenesis of Alzheimer's disease, and for this, the mass-production of amyloid-β peptide must be achieved first. Most of amyloid-β peptides commercially sold at present is produced by chemical synthetic methods such as solid-phase synthesis. There have been several attempts to optimize the synthetic condition to increase the production yield, but such chemical methods have the problem of poor yield due to such unique characteristics of amyloid-β peptide as insolubility and agglutinability. Recently, it has been tried to produce amyloid-β peptide by employing a genetic recombinant technique, but there still remain difficulties in achieving satisfactory production efficiency and stabilization of the recombinant protein produced.

The present inventors have therefore endeavored to solve such problems and develop a new genetic method for the production of recombinant amyloid-β peptide by fusing a gene encoding amyloid-β peptide at the C-terminus of a gene encoding ubiquitin, expressing the amyloid-β peptide in the form of a fusion protein with ubiquitin in a microorganism, purifying the fusion protein, separating the amyloid-β peptide from the fusion protein by treating with ubiquitin hydrolase and purifying the amyloid-β peptide. The method of the present invention is not hampered by the problems associated with the conventional chemical synthetic methods of amyloid-β peptide such as low production yield, high production cost and structural instability due to the insolubility and agglutinability of amyloid-β peptide, making it possible to efficiently produce amyloid-β peptide in large quantities.

### SUMMARY OF THE INVENTION

Accordingly, it is an object of the present invention to provide a method for producing a large quantity of amyloid-β peptide in a high yield by a genetic recombinant method.

### BRIEF DESCRIPTION OF THE DRAWINGS

The above and other objects and features of the present invention will become apparent from the following description of the invention, when taken in conjunction with the accompanying drawings, which respectively show:

FIG. [[1a]]1A: the amino acid sequence of the recombinant ubiquitin synthesized according to the present invention and the nucleotide sequence of the gene encoding the same;

FIG. [[1b]]1B: the procedure for constructing an expression vector comprising the gene encoding the recombinant ubiquitin of FIG. [[1a]]1A;

FIG. [[1c]]1C: the amino acid sequence of the recombinant amyloid-β peptide synthesized according to the present invention and the nucleotide sequence of the gene encoding the same;

FIG. [[1d]]1D: the procedure for constructing an expression vector expressing a ubiquitin-amyloid-β peptide fusion protein by cloning the recombinant amyloid-β peptide gene of FIG. [[1c]]1C into the expression vector of FIG. [[1b]]1B;

FIG. [[2a]]2A: the result of electrophoresis confirming the over-expression of a ubiquitin-amyloid-β peptide fusion protein from E. coli transformant obtained using the expression vector of FIG. [[1d]]1D;

−: without IPTG, +: with IPTG

FIG. [[2b]]2B: the result of affinity column chromatography for purifying the ubiquitin-amyloid-β peptide fusion protein over-expressed by E. coli transformant;

FIG. [[3a]]1A: the result of electrophoresis analysing the enzyme reactant obtained after treating the ubiquitin-amyloid-β peptide fusion protein with ubiquitin hydrolase and the amyloid-β peptide purified therefrom;

FIG. [[3b]]3B: the result of reverse phase chromatography for analyzing the ubiquitin-amyloid-β peptide fusion protein purified according to FIG. [[2b]]2B, the enzyme reactant obtained after the treatment of the fusion protein with ubiquitin hydrolase and the amyloid-β peptide purified therefrom;

FIGS. [[4a and 4b]]4A and 4B: the inhibitory effects on the cell activity of the recombinant amyloid-β peptide prepared according to the present invention as function of treatment concentration and time, respectively;

▴: genetic recombinant amyloid-β peptide (rAβ42)

●: recombinant ubiquitin (H₆Ub)

▪: ubiquitin-amyloid-β peptide fusion protein (H₆Ub-Aβ42)

□: chemically synthesized amyloid-β peptide (Aβ42)

FIG. [[4c]]4C: the inductive effect on apoptosis of the recombinant amyloid-β peptide prepared according to the present invention.

### DETAILED DESCRIPTION OF THE INVENTION

In accordance with one aspect of the present invention, there is provided a method for the production of amyloid-β peptide which comprises the steps of expressing and purifying the amyloid-β peptide from a microorganism in the form of a fusion protein with ubiquitin, separating the amyloid-β peptide from the fusion protein by treating with a ubiquitin-specific restriction enzyme and purifying the amyloid-β peptide from the enzyme reactant.

Hereinafter, the present invention is described in more detail.

The method of the present invention overcomes the difficulties encountered during the purification step due to the insolubility and agglutinability of amyloid-β peptide, and can provide amyloid-β peptide in large quantities without any change in the amino acid sequence thereof, which comprises the steps of:

1) preparing an expression vector comprising a fusion gene constructed by coupling a gene encoding amyloid-β peptide to the C-terminus of a gene encoding ubiquitin;

2) preparing a transformant by introducing the expression vector into a host cell;

3) allowing the transformant to express a fusion protein of amyloid-β peptide and ubiquitin and purifying the same;

4) treating the fusion protein with ubiquitin hydrolase to separate amyloid-β peptide therefrom; and

5) isolating the amyloid-β peptide.

In step 1), for the expression of amyloid-β peptide in the form of a fusion protein with ubiquitin, a fusion gene is constructed by coupling the gene encoding amyloid-β peptide to the C-terminus of the gene encoding ubiquitin and cloned into an expression vector.

Ubiquitin used as the fusion partner of amyloid-β peptide in the present invention is a relatively small protein consisting of 76 amino acids and found in all eukaryotic cells. Ubiquitin is synthesized not in the form of a monomer but in the form of an oligomer composed of several ubiquitin units linearly linked or in the form of a complex with a foreign protein connected to the C-terminus of ubiquitin. When such synthesized ubiquitin is treated with ubiquitin C-terminal hydrolase, monomeric ubiquitin involved in various intracellular processes is generated. The ubiquitin C-terminal hydrolase essential for the production of ubiquitin precisely cuts the peptide bond between ubiquitin and the protein regardless of the amino acid sequence or the structure of the protein connected to ubiquitin.

Meanwhile, ubiquitin and ubiquitin-relating enzymes including ubiquitin hydrolase are present in all eukaryotic cells, but not in prokaryotic cells such as bacteria. Therefore, when the gene encoding a fusion protein of a target protein and ubiquitin is expressed in prokaryotic cells, it is possible to produce the fusion protein in the form of ubiquitin connected to the target protein due to the lack of ubiquitin hydrolase, and to obtain the target protein by treating the fusion protein with ubiquitin hydrolase in vivo.

First, in order to prepare a fusion gene composed of a gene encoding amyloid-β peptide connected to the C-terminus of a gene encoding ubiquitin, a recombinant ubiquitin gene having the nucleotide sequence of SEQ ID NO: 9 is prepared by replacing some codons of the gene encoding ubiquitin with codons having high expression frequency in bacteria and introducing specific restriction enzyme sites to both ends thereof, which facilitates the cloning of the gene into a vector (see FIG. [[1a]]1A). The recombinant ubiquitin gene encodes the ubiquitin protein of the amino acid sequence of SEQ ID NO: 10. The recombinant ubiquitin gene thus prepared is cloned into an expression vector pre-treated with the restriction enzymes corresponding to the recognition sites introduced into both ends thereof (see FIG. [[1b]]1B). The expression vector employable in the present invention includes all vectors effective for the protein expression well-known in the art, but it is preferable to use a vector capable of expressing a fusion gene in prokaryotic cells having no ubiquitin hydrolase, in particular E coli expression vector. Further, it is preferable to employ an expression vector capable of introducing a 6-histidine-tag into the N-terminus of the recombinant ubiquitin gene, which facilitates the separation and purification of the expressed fusion protein. The procedure for cloning a gene into an expression vector can be conducted by appropriately combining DNA manipulation methods well-known in the art, such as restriction enzyme treatment, DNA ligation using a ligase, nucleotide synthesis using a DNA polymerase and so on.

The gene encoding amyloid-β peptide is synthesized by replacing some codons thereof with codons having high expression frequency in prokaryotic cells, according to the same method as described above. At this time, all amyloid-β peptides consisting of the 1^{st} to the 40^{th} amino acids, the 1^{st} to the 42^{nd} amino acids and the 1^{st} to the 43^{rd} amino acids in the amino acid sequence of SEQ ID NO: 11 can be employed as a template. The amyloid-β peptide gene is synthesized such that it contains the nucleotide sequence identical to the C-terminus of the ubiquitin gene having a specific restriction enzyme site at its N-terminus and has a termination codon (TAA) and another specific restriction enzyme site at its C-terminus (see FIG. [[1c]]1C). The recombinant amyloid-β peptide gene thus synthesized has the nucleotide sequence of SEQ ID NO: 18 and encodes the amyloid-β peptide protein having the amino acid sequence of SEQ ID NO: 19. The recombinant amyloid-β peptide gene is treated with the restriction enzymes corresponding to the recognition sites introduced into both ends thereof and cloned into a recombinant ubiquitin expression vector pre-treated with the same restriction enzymes, to prepare an expression vector comprising the ubiquitin-amyloid-β peptide fusion gene (see FIG. [[1d]]1D). The ubiquitin-amyloid-β peptide fusion gene cloned into the expression vector has the nucleotide sequence of SEQ ID NO: 20.

In step 2), the expression vector comprising the ubiquitin-amyloid-β peptide fusion gene prepared in step 1) is transformed into an appropriate host cell. The host cell employable in the present invention includes all host cells capable of operating the expression vector, and the transformation and selection of a transformant can be performed by employing an appropriate method among the previously known methods in the art according to the kinds and characteristics of the host cell and expression vector used. At this time, in order to obtain the amyloid-β peptide in the form of a fusion protein with ubiquitin, it is preferable to employ a prokaryotic cell as a host cell having no ubiquitin hydrolase, such as E. coli.

In step 3), the ubiquitin-amyloid-β peptide fusion protein is expressed by the transformant selected in step 2) and purified therefrom. When the transformant is cultured under the conditions suitable for expressing the fusion gene in the presence of an inducer, such as IPTG, the amyloid-β peptide is expressed in the form of a fusion protein with ubiquitin. After the electrophoresis and Coomassie staining, it has been confirmed that the ubiquitin-amyloid-β peptide fusion protein is over-expressed by the transformant (see FIG. [[2a]]2A).

For the purification of the ubiquitin-amyloid-β peptide fusion protein over-expressed by the transformant, the transformant cells are harvested from the culture solution, destroyed by ultrasonication, subjected the resulting solution to centrifugation to remove the supernatant containing soluble proteins, and then, the residual pellet is recovered. The pellet is mixed with a buffer containing 4 to 8 M urea to dissolve insoluble proteins and the resulting mixture was subjected to centrifugation to recover the supernatant.

The ubiquitin-amyloid-β peptide fusion protein over-expressed by the transformant according to the present invention has a 6-histidine-tag at its N-terminus, and accordingly, when the supernatant thus recovered is subjected to Ni-NTA affinity column chromatography, the fusion protein is adsorbed to the Ni-NTA resin. After urea is removed from the resin, the adsorbed fusion protein is eluted from the resin, subjected to electrophoresis, and then, confirmed by a Coomassie staining (see FIG. [[2b]]2B).

In step 4), the ubiquitin-amyloid-β peptide fusion protein purified in step 3) is treated with ubiquitin hydrolase, to separate the amyloid-β peptide from the fusion protein.

As described above, since prokaryotic cells lack ubiquitin hydrolase, the amyloid-β peptide expressed by the transformant is preserved in step 3) in the form of a fusion protein with ubiquitin. In order to cut the peptide bond between ubiquitin and amyloid-β peptide, the ubiquitin-amyloid-β peptide fusion protein purified in step 3) is treated with ubiquitin hydrolase at a concentration ranging from 1 to 10 μg/mg and reacted at 37□ for 1 to 3 hrs. The ubiquitin hydrolase employable in the present invention includes, but are not limited to, yeast ubiquitin hydrolase-1 (YUH-1) and ubiquitin hydrolase 41 (UBP41). The resulting enzyme reactant is subjected to electrophoresis and observed with a Coomassie staining. As a result, it has been confirmed that the binding site between ubiquitin and amyloid-β peptide in the fusion protein is precisely cut by the action of ubiquitin hydrolase and the fusion protein is separated into a ubiquitin fragment and an amyloid-β peptide fragment (see FIG. [[3a]]3A).

In step 5), for the purification of the amyloid-β peptide from the fusion protein, the enzyme reactant obtained in step 4) is subjected to reverse phase chromatography. The fraction corresponding to the elution time of the amyloid-β peptide is collected and all liquid ingredients thereof are removed by vaporization (see FIG. [[3b]]3B). The method of the present invention according to the above procedure can produce more than 3 mg of amyloid-β peptide from 3 g of E. coli transformant obtained from about 1 l of culture solution, in a yield of 12% or more.

The recombinant amyloid-β peptide prepared according to the present invention can suppress the cell activity and induce apoptosis to an extent which is equal to that achievable with chemically synthesized amyloid-β peptide (see FIGS. [[4a to 4c]]4A to 4C).

Accordingly, the method of the present invention overcomes the problems associated with the chemical synthesis of amyloid-β peptide such as low production yield, high production cost and handling difficulties arising from the insolubility and agglutinability of amyloid-β peptide, and it can be effectively used for the mass-production of amyloid-β peptide.

The following Examples are intended to further illustrate the present invention without limiting its scope.

### EXAMPLE 1

### Cloning of a Ubiquitin-Amyloid-β Peptide Fusion Gene

In order to prepare an expression vector comprising a ubiquitin-amyloid-β peptide fusion gene, a protein expression vector containing a recombinant ubiquitin gene was prepared first.

In order to enhance the expression of the gene encoding ubiquitin in bacteria, codons having high expression frequency were introduced thereto. First, four pairs of 8 oligonucleotides having the nucleotide sequences of SEQ ID NOs: 1 to 8 (fUb1, fUb2, fUb3, fUb4, rUb1, rUb2, rUb3 and rUb4) were synthesized. Two oligonucleotides having nucleotide sequences complementary to each other (fUb1+rUb1, fUb2+rUb2, fUb3+rUb3 and fUb4+rUb4) were subjected to complementary binding to generate four double-strand fragments Ub1, Ub2, Ub3 and Ub4. Subsequently, the Ub1-Ub2, and Ub3-Ub4 pairs were each subjected to ligation, and the resulting two fragments were subjected to ligation again, to obtain a full-length ubiquitin gene. Each of the above ligation reactions was conducted at 16□ for 16 hrs after 500 ng of each oligonucleotide and 5 units of T4 DNA ligase were mixed with 20 μl of a ligation buffer. The recombinant ubiquitin gene thus obtained has the nucleotide sequence of SEQ ID NO: 9. Further, as shown in FIG. [[1a]]1A, it was synthesized to have 5′-C-3′ at its C-terminus, the configuration generated when treated with reatriction enzyme XhoI and 5′-GATCC-3′ at its N-terminus, the configuration generated when treated with restriction enzyme BamHI. The recombinant ubiquitin gene having recognition sites for BamHI and XhoI at both ends were directly cloned into vector pET28a (Novagen) pre-treated with BamHI and XhoI, to obtain a recombinant ubiquitin expression vector, pET/H₆Ub (FIG. [[1b]]1B). The recombinant ubiquitin expressed from the expression vector pET/H₆Ub contained a 6-histidine tag at its N-terminus and had the amino acid sequence of SEQ ID NO: 10. Further, the C-terminus of the recombinant ubiquitin gene (215-220 bp) was designed to contain the recognition site (CTTAAG) for restriction enzyme AflII, which facilitated the following cloning procedure.

Meanwhile, the gene encoding amyloid-beta42 peptide which consists of the 1^{st} to the 42^{nd} amino acid sequences in the amino acid sequence of SEQ ID NO: 11 was synthesized by employing three pairs of 6 oligonucleotides having the nucleotide sequences of SEQ ID NOs: 12 to 17 (fAβ1, fAβ2, fAβ3, rAβ1, rAβ2 and rAβ3), according to the same method as described above (FIG. [[1c]]1C). Two oligonucleotides having nucleotide sequences complementary to each other (fAβ1+rAβ1, fAβ2+rAβ2, fAβ3+rAβ3) were subjected to complementary binding, to generate three double-strand fragments Aβ1, Aβ2 and Aβ3. Two fragments Aβ1 and Aβ2 were subjected to ligation, and then, the resulting fragment was ligated to the remaining fragment Aβ3, to generate a recombinant amyloid-β peptide gene. The recombinant amyloid-β peptide gene thus generated had the nucleotide sequence of SEQ ID NO: 18, which encodes a recombinant amyloid-beta peptide having the amino acid sequence of SEQ ID NO: 19. As shown in FIG. [[1 c]]1C, the 5′-end of the recombinant amyloid-β peptide gene contained 5′-TTAAG-3′, which is the configuration generated when treated with AflII, as well as 5′-ACTGCGTGGCGGC-3′, which is identical to the nucleotide sequence of the residual ubiquitin C-terminus shown in FIG. [[1a]]1A; and the 3′-end thereof, a termination codon (TAA) and 5′-C-3′, the configuration generated when treated with XhoI. The recombinant amyloid-β peptide gene having such modified ends was inserted into a ubiquitin expression vector pET/H₆Ub pre-treated with AflII and XhoI, to obtain a ubiquitin-amyloid-β peptide expression vector, pET/H₆Ub-Aβ42 (FIG. [[1d]]1D). Sequencing analysis showed that the ubiquitin-amyloid-β peptide fusion gene cloned into the expression vector had the nucleotide sequence of SEQ ID NO: 20.

### EXAMPLE 2

### Expression and Purification of a Ubiquitin-Amyloid-β Peptide Fusion Protein

E. Coli BL21(DE3) cells (Novagen) were transformed with the ubiquitin-amyloid-β peptide fusion gene prepared in Example 1, suspended in 1 ml of LB broth and cultured at 37□ for an hour. The cultured cells were spread on LB agar plate containing 30 μg/ml of kanamycin to select a transformant introduced with the ubiquitin-amyloid-β peptide fusion gene. The transformant thus selected was inoculated into LB broth containing 30 μg/ml of kanamycin and cultured at 37□ for 4 hrs with shaking. ITPG (1 mM) was added to the culture solution and the cells were further cultured at the same temperature for 3 hrs. The resulting culture solution was subjected to centrifugation at 5,000×g for 20 min to recover a cell pellet. At this time, the E. coli transformed with the expression vector pET/H₆Ub comprising the recombinant ubiquitin gene was employed as a control.

In order to assess the level of expression of the ubiquitin-amyloid-β peptide fusion protein by the E. coli transformant, the cell pellet thus recovered was dissolved in 4% SDS, subjected to electrophoresis, and stained with Coomassie blue. As a result, in case of inducing protein expression with IPTG (+), a distinctive band was detected at a position of about 17 kDa, which confirms that the ubiquitin-amyloid-β peptide fusion protein was over-expressed by the above E. coli transformant (FIG. [[2a]]2A). A protein band detected at a position of about 10 kDa in FIG. [[2a]]2A was ubiquitin expressed by the control transformant.

In order to purify the ubiquitin-amyloid-β peptide fusion protein expressed by the E coli transformant, 3 g of the cell pellet separated from the culture solution was suspended in 15 ml of buffer A (50 mM Tris, 150 mM NaCl, pH 8.0) and subjected to ultrasonication, to obtain a homogeneous cell solution. The homogeneous cell solution was subjected to centrifugation at 10,000×g for 30 min to separate the supernatant containing soluble proteins and an insoluble pellet. Since the ubiquitin-amyloid-β peptide fusion protein was insoluble, the supernatant was removed and the pellet obtained after the above centrifugation was recovered. The insoluble pellet thus recovered was suspended in 15 ml of buffer B, which was prepared by adding 8 M urea to buffer A, stirred at room temperature for an hour, and then, subjected to centrifugation at 10,000×g for 30 min to separate the supernatant from the undissolved pellet.

The supernatant thus obtained was mixed with 0.5 ml of Ni-NTA affinity resin (Novagen) at 4□ for 3 hrs to allow the 6-histidine tagged ubiquitin-amyloid-β peptide fusion protein to bind with the Ni-NTA resin. The resin was washed with 10 ml of buffer A, subjected to centrifugation at 1,500 rpm for 5 min to precipitate the Ni-NTA resin, and the supernatant was removed. The above procedure was repeated three times. The Ni-NTA resin having the fusion protein adsorbed was washed with 2 ml of buffer C, which was prepared by adding 50 mM of imidazole to buffer A, to remove the residual non-specifically bound component, and the ubiquitin-amyloid-β peptide fusion protein was eluted from the Ni-NTA resin by using buffer D which was prepared by adding 400 mM of imidazole to the buffer A. The fraction eluted therefrom was subjected to electrophoresis and stained with Commassie blue. As a result, a ubiquitin-amyloid-β peptide fusion protein of about 17 kDa was recovered in a highly purified form from the transformant culture solution (FIG. [[2b]]2B).

### EXAMPLE 3

### Separation and Purification of Amyloid-β Peptide from a Ubiquitin-Amyloid-β Peptide Fusion Protein

Since prokaryotic cells such as E. coli do not have any ubiquitin hydrolase, the ubiquitin-amyloid-β peptide was purified in the form of a fusion protein from the E. coli transformant. In order to separate the amyloid-β peptide form the fusion protein, yeast ubiquitin hydrolase-1 (YUH-1) capable of specifically cutting the peptide bond between ubiquitin and amyloid-β peptide in the fusion protein was cloned, expressed and purified according to the common recombinant method in the art (Protein Expression and Purification 40: 183-189, 2005). The ubiquitin-amyloid-β peptide fusion protein purified in Example 2 was treated with 3 μg/mg of YUH-1 and reacted at 37□ for 2 hrs to cut the binding site between ubiquitin and amyloid-β peptide in the fusion protein. In order to purify only the amyloid-β peptide, the reaction mixture was subjected to reverse phase chromatography using POROS R2 column containing C8 resin (Applied Biosystems). At this time, before the loading onto the column, the reaction mixture was acidified with 10% acetic acid, and the resulting column was subjected to elution using the mixture of solution A (0.05% trifluoroacetic acid) and solution B (0.05% trifluoroacetic acid and 90% acetonitrile) while gradually increasing the concentration of solution B. The amyloid-β peptide begun to elute at the point when the concentration of solution B reached to 33%, and the eluting time of the amyloid-β peptide from the column was 7.5 min. The fraction at that elution time was collected and subjected to vaporization to remove all liquid components, to obtain the amyloid-β peptide.

FIG. [[3a]]3A shows the result of electrophoresis analyzing the samples obtained in each step for separating the amyloid-β peptide from the fusion protein by the action of ubiquitin hydrolase, wherein lane 1 is the purified ubiquitin-amyloid-β peptide fusion protein; lane 2, the reactant treated with ubiquitin hydrolase; and lane 3, the purified amyloid-β peptide from the above reactant.

FIG. [[3b]]3B shows the result of reverse phase chromatography analyzing the samples obtained in each step of FIG. [[3a]]3A, which confirms that the amyloid-β peptide was eluted at 7.5 min. According to the above purification procedure, about 3 mg or more of the amyloid-β peptide was recovered from 3 g of the E. coli transformant which was harvested from about 1 l of the culture solution. Accordingly, the overall production yield was more than 12%.

### EXAMPLE 4

### Physiological Activity of a Genetic Recombinant Amyloid-β Peptide

To compare the physiological activity of the genetic recombinant amyloid-β peptide produced according to the present invention with that of the amyloid-β peptide produced by a chemical synthetic method, their mitochondrial activities in eykaryotic cells were examined as follows:

First, 0.45 mg of the amyloid-β peptide prepared in Example 3 was dissolved in 20 μl of dimethyl sulfoxide, and 980 μl of DMEM was added thereto, to prepare the amyloid-β peptide solution at a concentration of 100 μM, which was stored at 4□ for 24 hrs and subjected to centrifugation to separate the supernatant from the precipitate. The supernatant was distributed to an eppendorf tube at a volume of 50 μl and stored at −80□.

Human neuroblastoma SH-SY5Y cell line (ATCC CRL-2266) was suspended in DMEM containing 10% FBS, distributed to each well of a 96-well plate at a concentration of 7×10³ cells/well, and then, cultured at 37□ for 24 hrs. The genetic recombinant amyloid-β peptide solution prepared above was added to each well at a concentration of 1, 2 or 4 μM, and the well plate was incubated at 37□ for 24 hrs. The cell activity was measured by means of MTT (3-(4,5-dimethylthiazol-2-yl)-2,5-diphenyltetrazolium bromide) assay which is capable of measuring the in vivo mitochondria dehydrogenase activity. MTT was added to each well at a concentration of 0.8 mg/ml, and the reactants in the well were allowed to react at 37□ for 5 hrs to induce in vivo dehydrogenase reaction of mitochondria. Formazan crystals generated from such reaction were dissolved in dimethyl sulfoxide and its adsorbance was measured at 550 nm.

FIG. [[4a]]4A shows the degree of suppression of the cell activity by the recombinant amyloid-β peptide (rAβ42: ▴) concentration. Also shown in FIG. [[4a]]4A are the results obtained using the recombinant ubiquitin (H₆Ub: ●) control, the ubiquitin-amyloid-β peptide fusion protein (H₆Ub-Aβ42: ▪) and the chemically synthesized amyloid-β peptide (Aβ42: □). From these results, it has been found that the genetic recombinant amyloid-β peptide according to the present invention can suppress the cell activity just like the chemically synthesized amyloid-β peptide.

FIG. [[4b]]4B shows the result of measuring the changes in the cell activity caused by the genetic recombinant amyloid-β peptide prepared of the present invention according to the treatment time. When the cells were treated with 5 μM of the amyloid-β peptide for 12, 24 and 48 hrs, respectively, as the treatment time becomes longer, the cell activity becomes further reduced.

FIG. [[4c]]4C shows the result of measuring the level of apoptosis caused by the recombinant amyloid-β peptide prepared according to the present invention by the method of trypan blue staining. After the cells were treated with 5 μM of the amyloid-β peptide for 36 hrs, black cells stained with trypan blue were examined to determine the level of apoptosis induced. As a result, it has been found that the recombinant amyloid-β peptide (rAβ42) showed an apoptotic activity, which was significantly higher than the recombinant ubiquitin (H₆Ub) or ubiquitin-amyloid fusion protein (H₆Ub-Aβ42), the activity being equal to, or slightly higher than, the chemically synthesized amyloid-β peptide (Aβ42).

While the invention has been described with respect to the above specific embodiments, it should be recognized that various modifications and changes may be made to the invention by those skilled in the art which also fall within the scope of the invention as defined by the appended claims.

What is claimed is:

1. A method for the preparation of amyloid-β peptide which comprises the steps of:
1) preparing an expression vector comprising a fusion gene constructed by coupling a gene encoding amyloid-β peptide to the C-terminus of a gene encoding ubiquitin;
2) preparing a transformant by introducing the expression vector into a host cell;
3) allowing the transformant to express a fusion protein of amyloid-β peptide and ubiquitin encoded by the fusion gene and purifying the expressed fusion protein;
4) treating the fusion protein with ubiquitin hydrolase to separate amyloid-β peptide from the fusion protein; and
5) isolating the amyloid-β peptide from the reaction mixture obtained in step 4).

2. The method of claim 1, wherein the amyloid-β peptide of step 1) consists of 28 to 43 amino acids selected from the amino acid sequence of SEQ ID NO: 11 starting from the N-terminus thereof.

3. The method of claim 1, wherein the expression vector of step 1) is an expression vector capable of expressing a target protein in a prokaryotic cell.

4. The method of claim 1, wherein the host cell of step 2) is a prokaryotic cell.

5. The method of claim 1, wherein the fusion protein of step 3) contains the amyloid-β peptide at the C-terminus of ubiquitin and a 6-histidine tag at the N-terminus thereof.

6. The method of claim 1, wherein the fusion protein obtained in step 3) is purified by the steps of:
1) harvesting the transformant culture solution, destroying the cells, and centrifuging the resulting solution to obtain a pellet fraction;
2) adding a buffer containing 4 to 8 M urea to the pellet fraction to dissolve insoluble proteins and centrifuging the resulting solution to separate a supernatant;
3) loading the supernatant onto an Ni-NTA affinity column using a buffer containing urea so that the fusion protein is adsorbed to the Ni-NTA resin;
4) removing urea from the column leaving the fusion protein adsorbed to the Ni-NTA resin; and
5) eluting the adsorbed fusion protein from the Ni-NTA resin by using a buffer containing imidazole.

7. The method of claim 1, wherein the amyloid-β peptide obtained in step 5) is purified by reverse phase chromatography.


SEQUENCE LISTING (20 sequences)

SEQ ID NO: 1 - DNA, 42 bp, Artificial Sequence
  fUb1
gatccatgcagatcttcgtgaaaaccctgaccggcaaaacca

SEQ ID NO: 2 - DNA, 56 bp, Artificial Sequence
  fUb2
tcactctagaagtggaaccgagcgataccatcgaaaacgtgaaagcgaaaatccag

SEQ ID NO: 3 - DNA, 66 bp, Artificial Sequence
  fUb3
gataaagaaggcatcccgccggatcagcagcgtctgatctttgcgggcaaacagctggaagatggc

SEQ ID NO: 4 - DNA, 73 bp, Artificial Sequence
  fUb4
cgcaccctgtctgattataacatccagaaagagtcgaccctgcatctggtcttaagactgcgtggcggctaac

SEQ ID NO: 5 - DNA, 47 bp, Artificial Sequence
  rUb1
ctagagtgatggttttgccggtcagggttttcacgaagatctgcatg

SEQ ID NO: 6 - DNA, 56 bp, Artificial Sequence
  rUb2
ttctttatcctggattttcgctttcacgttttcgatggtatcgctcggttccactt

SEQ ID NO: 7 - DNA, 69 bp, Artificial Sequence
  rUb3
agacagggtgcggccatcttccagctgtttgcccgcaaagatcagacgctgctgatccggcgggatgcc

SEQ ID NO: 8 - DNA, 65 bp, Artificial Sequence
  rUb4
tcgagttagccgccacgcagtcttaagaccagatgcagggtcgactctttctggatgttataatc

SEQ ID NO: 9 - DNA, 237 bp, Artificial Sequence
  rUb
gatccatgcagatcttcgtgaaaaccctgaccggcaaaaccatcactctagaagtggaaccgagcgataccatcgaaaacgtgaaagcgaaaatccaggataaagaaggcatcccgccggatcagcagcgtctgatctttgcgggcaaacagctggaagatggccgcaccctgtctgattataacatccagaaagagtcgaccctgcatctggtcttaagactgcgtggcggctaac

SEQ ID NO: 10 - protein, 76 aa, Artificial Sequence [sequence omitted]

SEQ ID NO: 11 - protein, 42 aa, Artificial Sequence [sequence omitted]

SEQ ID NO: 12 - DNA, 39 bp, Artificial Sequence
  fAb1
ttaagactgcgtggcggcgatgcggaatttcgacatgac

SEQ ID NO: 13 - DNA, 69 bp, Artificial Sequence
  fAb2
tcaggatatgaagttcatcatcaaaaattggtgttctttgcagaagatgtgggttcaaacaaaggtgca

SEQ ID NO: 14 - DNA, 40 bp, Artificial Sequence
  fAb3
atcattggactcatggtgggcggcgttgttattgcgtaac

SEQ ID NO: 15 - DNA, 54 bp, Artificial Sequence
  rAb1
gatgaacttcatatcctgagtcatgtcgaaattccgcatcgccgccacgcagtc

SEQ ID NO: 16 - DNA, 57 bp, Artificial Sequence
  rAb2
caatgattgcacctttgtttgaacccacatcttctgcaaagaactccaatttttgat

SEQ ID NO: 17 - DNA, 37 bp, Artificial Sequence
  rAb3
tcgagttacgcaataacaacgccgcccaccatgagtc

SEQ ID NO: 18 - DNA, 148 bp, Artificial Sequence
  rAb42
ttaagactgcgtggcggcgatgcggaatttcgacatgactcaggatatgaagttcatcatcaaaaattggtgttctttgcagaagatgtgggttcaaacaaaggtgcaatcattggactcatggtcggcggtgttgttattgcgtaac

SEQ ID NO: 19 - protein, 48 aa, Artificial Sequence
  rAb42
LRLRGGDAEFRHDSGYEVHHQKLVFFAEDVGSNKGAIIGLMVGGVVIA

SEQ ID NO: 20 - DNA, 357 bp, Artificial Sequence
  Ub-Ab42
atgcagatcttcgtgaaaaccctgaccggcaaaaccatcactctagaagtggaaccgagcgataccatcgaaaacgtgaaagcgaaaatccaggataaagaaggcatcccgccggatcagcagcgtctgatctttgcgggcaaacagctggaagatggccgcaccctgtctgattataacatccagaaagagtcgaccctgcatctggtcttaagactgcgtggcggcgatgcggaatttcgacatgactcaggatatgaagttcatcatcaaaaattggtgttctttgcagaagatgtgggttcaaacaaaggtgcaatcattggactcatggtcggcggtgttgttattgcgtaa